(19) United States
(12) Patent Application Publication
(10) Pub. No.: US 2026/0334799 A1
(43) Pub. Date: Oct. 29, 2026

(54) CUFFLESS BLOOD PRESSURE MONITOR PATCH AND METHOD FOR PLACING THE CUFFLESS BLOOD PRESSURE MONITOR PATCH

(71) Applicant: STMicroelectronics International N.V., Geneva (CH)

(72) Inventors: Mahesh CHOWDHARY, San Jose, CA (US); Swapnil Sayan SAHA, Mountain View, CA (US)

(73) Assignee: STMicroelectronics International N.V., Geneva (CH)

(21) Appl. No.: 19/191,993

(22) Filed: Apr. 28, 2025

(51) Int. Cl. A61B 5/021; A61B 5/00

(52) U.S. Cl. CPC A61B 5/02141; A61B 5/6822; A61B 5/6831; A61B 5/6832; A61B 5/684; A61B 5/7235; A61B 5/741; A61B 5/742; A61B 2560/0462; A61B 2562/0219

(57) ABSTRACT

A blood pressure monitoring device includes a patch including two inertial measurement units placed adjacent to the skin of a user. The blood pressure monitoring device is configured to transmit sensor data to an electronic device. The electronic device is configured to guide the user through a series of positional adjustments to align the two inertial measurement units along an artery of the user based on the sensor data.

### BACKGROUND

### Technical Field

The present disclosure is related to blood pressure monitors, and more particularly, to cuffless blood pressure monitors.

### Description of the Related Art

Blood pressure can be a useful metric for individuals in a variety of situations. For example, it can be beneficial for individuals with hypertension to monitor their blood pressure periodically in order to avoid serious medical issues. It can also be beneficial to monitor the blood pressure of individuals when exercising for fitness purposes. It may be beneficial to monitor the blood pressure of individuals for a variety of other reasons.

Typically, blood pressure is measured at a physician's office. In these cases, a blood pressure monitoring cuff is wrapped around an individual's arm. The blood pressure cuff includes an inflatable bladder and a hose. The inflatable bladder is inflated to an air pressure sufficient to prevent blood flow in a local main artery. The inflatable bladder is gradually deflated until blood begins to flow through the artery. A pressure sensor measures the pressure at which the blood begins to flow. This corresponds to the systolic blood pressure. The pressure measurement is also taken when blood flow is no longer restricted. This corresponds to the diastolic blood pressure.

While a blood pressure cuff is an effective way of measuring blood pressure, there are several drawbacks. The standard upper arm inflatable sphygmomanometer offers discrete measurements, is not comfortable or portable, and is dependent on proper cuff placement and cuff size. Arterial catheterization is the gold standard and is continuous but invasive. Cuffless devices provide a chance to address these limitations.

A cuffless blood pressure device can address some of drawbacks of blood pressure cuffs. Among the various methods for cuffless blood pressure monitors, pulse transit time (PTT) is the most popular due to robust theoretical principles, a broad spectrum of supported sensors, and flexible sensor locations. However, there are also difficulties faced by cuffless blood pressure devices. Firstly, the model parameters are person-specific and evolve with age. Heuristic approaches and extensive feature engineering are called for to model these parameters. Approximations of these parameters are affected by motion artifacts and interference from superficial blood vessels, calling for multiple electrode placement, a complex signal processing chain, and higher power consumption. Machine learning models tunes these parameters using data driven approach, but need cuff calibration and proper patch placement to work.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which, in and of itself, may also be inventive.

### BRIEF SUMMARY

Embodiments of the present disclosure provide an accelerometer-based cuffless blood pressure monitoring device that can effectively and efficiently monitor the blood pressure of an individual. Embodiments of the present disclosure provide a method for effectively and efficiently aligning the cuffless blood pressure monitor. While the description herein is directed primarily toward a cuffless blood pressure monitoring device based on PTT and ballistocardiography, embodiments of the present disclosure extend to biosensors other than blood pressure monitoring devices and corresponding alignment methods.

In one embodiment, the blood pressure monitoring device includes a first inertial measurement unit and a second inertial measurement unit. The first and second inertial measurement units are spaced apart from each other by a fixed distance in a sensor housing. During use of the blood pressure monitoring device, the blood pressure monitoring device is placed on the skin of the user. The first and second inertial measurement units generate sensor data based on the flow of blood through a vein or artery over which the first and second inertial measurement units are positioned. As the blood pressure measurement is most accurate when the first and second inertial measurement units are each positioned directly over the vein or artery, the blood pressure measurement device includes a handle or other adjustment mechanism that enables the user to adjust the position of the sensor housing with respect to the rest of the blood pressure monitoring device.

In one embodiment, the handle enables the user to translate the position of the sensor housing along a first axis (e.g., left/right). In one embodiment, the handle enables the user to translate the position of the sensor housing along a second axis (e.g., up/down) perpendicular to the first axis. In one embodiment, the handles enables the user to rotate the position of the housing clockwise (CW) or anticlockwise (AW) around a third axis perpendicular to the first and second axes.

In one embodiment, the blood pressure monitoring device includes a wireless transceiver that enables the blood pressure monitoring device to transmit sensor data generated by the first and second inertial measurement units to a nearby electronic device paired to the blood pressure monitoring device. The electronic device generates a blood pressure measurement based on the sensor data.

In one embodiment, a method assists the user to align the sensor housing preparatory to performing a blood pressure measurement. The user places the blood pressure monitoring device at a selected location on the body (e.g., the right side of the neck over the carotid artery). The first and second inertial measurement units each generate sensor data. The blood pressure monitoring device transmits the sensor data to the electronic device. The electronic device analyzes the sensor data and outputs an instruction to the user to adjust the handle in a selected manner. The blood pressure monitoring device transmits sensor data from the new position. The electronic device again analyzes the sensor data and outputs another instruction to the user to make a selected adjustment to the handle. The process of adjustment instructions and analysis continues until the electronic device identifies an acceptable (or optimal) position. The blood pressure monitoring patch is now aligned with the vein or artery. The electronic device receives sensor data from the blood pressure monitoring device at the aligned position and generates a blood pressure measurement. The electronic device outputs the blood pressure measurement to the user.

In one embodiment, a method includes generating first sensor data and second sensor data with a first inertial measurement unit and a second inertial measurement unit of a biosensor patch positioned on a skin of a user, transmitting the first sensor data and the second sensor data to an electronic device, and analyzing the first sensor data and the second sensor data with the electronic device. The method includes identifying, with the electronic device, an acceptable position of the patch by instructing the user to make a plurality of positional adjustments of the patch based on the first sensor data and the second sensor data. The method includes outputting a biometric measurement based on the first sensor data and the second sensor data at the acceptable position.

In one embodiment, a method includes receiving, with an electronic device, sensor data from a biosensor positioned on a skin of a user, analyzing the sensor data with the electronic device, and outputting from the electronic device instructions to the user including a plurality of positional adjustments to be made to the biosensor based on the sensor data. The method includes identifying, with the electronic device, an acceptable position of the biosensor based on the sensor data and the positional adjustments and outputting a biometric measurement based on the first sensor data and the second sensor data at the acceptable position.

In one embodiment, a blood pressure monitoring device includes a sensor housing, a first inertial measurement unit in the sensor housing, and a second inertial measurement unit in the sensor housing spaced apart from the first inertial measurement unit by a fixed distance. The device includes an adjustment mechanism mechanically coupled to the sensor housing and configured to rotate the sensor housing around a first axis and to translate the sensor housing along a second axis transverse to the first axis.

### BRIEF DESCRIPTION OF THE SEVERAL VIEWS OF THE DRAWINGS

Reference will now be made by way of example only to the accompanying drawings. In the drawings, identical reference numbers identify similar elements or acts. In some drawings, however, different reference numbers may be used to indicate the same or similar elements. The sizes and relative positions of elements in the drawings are not necessarily drawn to scale. For example, the shapes of various elements and angles are not necessarily drawn to scale, and some of these elements may be enlarged and positioned to improve drawing legibility.

FIG. 1 is a block diagram of a blood pressure monitoring system, according to one embodiment.

FIGS. 2A-2C are illustrations of a cuffless blood pressure monitoring device, according to one embodiment.

FIGS. 3A-3M illustrate positional adjustments of a handle/sensor housing of a blood pressure monitoring device, according to one embodiment.

FIG. 4 is an illustration of a user utilizing a blood pressure monitoring system, according to one embodiment.

FIGS. 5A and 5B are graphs illustrating sensor data from first and second inertial measurement units of a cuffless blood pressure monitoring device, according to one embodiment.

FIG. 6 is a flow diagram of a method for aligning a blood pressure monitor, according to one embodiment.

FIG. 7 is a functional flow diagram illustrating processing of sensor data in a blood pressure monitoring system, according to one embodiment.

FIGS. 8A and 8B are a flow diagram of a method for aligning a blood pressure monitor, according to one embodiment.

FIG. 9 is a flow diagram of a method for aligning a blood pressure monitor, according to one embodiment.

FIGS. 10A-10H of the positions of first and second inertial measurement units relative to an artery, according to one embodiment.

FIG. 11 is a graph indicating sensor data values for various inertial measurement unit positions relative to an artery, in accordance with one embodiment.

FIG. 12 is a block diagram of an electronic device, according to one embodiment.

FIG. 13 is a block diagram of a blood pressure monitoring device, according to one embodiment.

FIG. 14 is an illustration of an electronic device during an alignment process of a blood pressure monitor, according to one embodiment.

FIG. 15 is a flow diagram of a method for aligning a blood pressure monitor, according to one embodiment.

FIG. 16 is a flow diagram of a method for aligning a blood pressure monitor, according to one embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known systems, components, and circuitry associated with integrated circuits have not been shown or described in detail, to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.” Further, the terms “first,” “second,” and similar indicators of sequence are to be construed as interchangeable unless the context clearly dictates otherwise.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

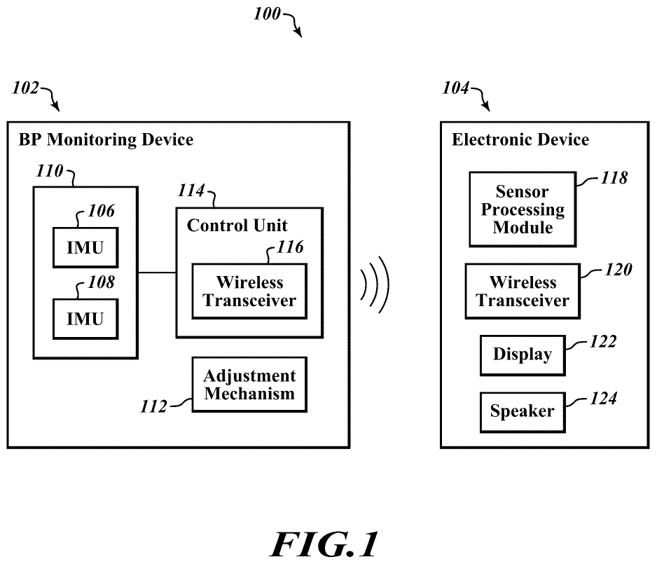

FIG. 1 is a block diagram of a blood pressure monitoring system 100, according to one embodiment. The blood pressure monitoring system 100 includes a blood pressure monitoring device 102 and an electronic device 104. As will be set forth in more detail below, the blood pressure monitoring device 102 and the electronic device 104 cooperate to perform an alignment process of the blood pressure monitoring device 102 and to generate a blood pressure measurement.

While the description and figures focus primarily on a blood pressure monitoring system, embodiments of the present disclosure also extend to biosensor systems other than blood pressure monitoring systems. The biosensor systems include a biosensor device placed on a user. Each biosensor device includes one or more sensors and a handle or adjustment mechanism that facilitates calibration or alignment of the biosensor. While this disclosure is directed primarily to an accelerometer sensor, other biosensors may also be used. Examples of other biosensors include blood oxygen sensors, heart rate sensors, EKG sensors, ECG sensors, or other types of biosensors.

The blood pressure monitoring device 102 is configured to be placed on the skin of a user adjacent to a vein or artery of the user. In a primary example described herein, the blood pressure monitoring device 102 is configured to be placed on the right side of the neck over the carotid artery of the user. However, other placement locations can be utilized without departing from the scope of the present disclosure.

In one embodiment, the blood pressure monitoring device 102 is a cuffless blood pressure monitoring device. Accordingly, the blood pressure monitoring device 102 does not include an inflatable bladder that wraps around an arm of the user. Instead, the blood pressure monitoring device 102 includes at least a portion that is placed adjacent to the skin of the user. For example, a portion of the blood pressure monitoring device may be placed adjacent to a selected artery of the user. The inertial measurement units 106 and 108 are close to the skin of the user. The inertial measurement units 106 and 108 sense vibration or motion of the skin based on the flow of blood through the artery. The inertial measurement units generate sensor signals based on the sensed motion.

In one embodiment, the blood pressure monitoring device 102 includes a sensor housing 110. The inertial measurement units 106 and 108 are positioned within the housing 110. The sensor housing includes a substrate on which the inertial measurement units 106 and 108 are positioned. In one embodiment, the inertial measurement units 106 and 108 are spaced apart from each other by a fixed distance. In some embodiments, the fixed distance is between 1 cm and 2 cm, though other separation distances can be utilized without departing from the scope of the present disclosure.

In one embodiment, the sensor housing 110 encloses the inertial measurement units 106 and 108. The sensor housing can include a cover or lid that covers the inertial measurement units 106 and 108 such that the inertial measurement units 106 and 108 are positioned between the substrate and the lid or cover.

In one embodiment, the blood pressure monitoring device 102 includes an adjustment mechanism 112. In one embodiment, the adjustment mechanism is mechanically coupled to the sensor housing 110. Movement of the adjustment mechanism results in corresponding movement of the sensor housing. As the inertial measurement units 106 and 108 are positioned within the sensor housing 110, movement of the sensor housing results in movement of the inertial measurement units 106 and 108.

In one embodiment, the adjustment mechanism 112 is able to translate (e.g., move/shift) back and forth along a first axis. In one example, the first axis may be considered as an X-axis to left and right directions. In one embodiment, the adjustment mechanism is able to translate back and forth along a second axis transverse to the first axis. In one example, the second axis may be considered a Y-axis corresponding substantially to up-and-down directions. In one example, the adjustment mechanism 112 is able to rotate clockwise and anticlockwise around a third axis transverse to the first axis and the second axis. In one embodiment, the third axis may be considered a Z-axis pointing outward from the user's skin. In one embodiment, the first axis, the second axis, and the third axis are mutually perpendicular to each other.

In one embodiment, the adjustment mechanism 112 is a handle coupled to the sensor housing 110. In one embodiment the adjustment mechanism includes one or more of a grip, a knob, a protrusion, or another type of member configured to be manipulated by the fingers of a user. In one embodiment, the device housing itself is the adjustment mechanism. Other types of adjustment mechanisms can be utilized without departing from the scope of the present disclosure.

In one embodiment, the control unit 114 corresponds to circuitry positioned within a main housing of the blood pressure monitoring device 102. The control unit 114 is communicatively coupled to the inertial measurement units 106 and 108. The control unit 114 is coupled to the inertial measurement units 106 and 108 by one or more wires, cords, signal traces, or other couplings that can carry electrical signals the inertial measurement units 106 and 108 and the control unit 114. Or cable may be coupled the first portion of the blood pressure monitoring device 102 to the control unit 114.

In one embodiment, the control unit 114 is in a same housing as the inertial measurement units 106 and 108. Various configurations of the blood pressure monitoring device 102 can be utilized without departing from the scope of the present disclosure.

In one embodiment, the inertial measurement unit 106 includes an accelerometer. The accelerometer can include a single axis accelerometer or a multiaxes accelerometers. In one embodiment, the accelerometer is a three-axis accelerometer that senses acceleration in three mutually orthogonal axes. The accelerometer can be sensitive to vibrations or motion of the skin resulting from blood flow through the artery adjacent to the inertial measurement unit 106.

In one embodiment, the inertial measurement unit 106 includes a gyroscope. The gyroscope can include a single axis gyroscope or a multiaxes gyroscopes. In one embodiment, the gyroscope is a three-axis gyroscope that senses rotation about three mutually orthogonal axes. In one embodiment, the inertial measurement unit 106 includes both an accelerometer and gyroscope.

The inertial measurement unit 108 can include the same types of devices as the inertial measurement unit 106. In particular, the inertial measurement unit 108 can include an accelerometer, a gyroscope, or both of accelerometer and gyroscope. In one embodiment, the inertial measurement unit 108 is substantially identical to the inertial measurement unit 106 except that the inertial measurement unit 108 is separated from the inertial measurement unit 106 by a selected distance. As will be described in more detail below, the selected distance enables the inertial measurement unit 106 to generate sensor signals with a timing offset relative to the inertial measurement unit 108. This can assist in generating an estimation of the blood pressure. In a particular example described here, the inertial measurement unit 106 and the inertial measurement unit 108 each have a three-axis accelerometer.

In one embodiment, the inertial measurement units 106 and 108 generate sensor signals. The sensor signals are indicative of accelerations or other movements sensed by the inertial measurement units 106 and 108. As will be described in more detail below, the control unit 114 utilizes the sensor signals to predict or estimate blood pressure. In one embodiment, the inertial measurement units 106 and 108 generate analog sensor signals and provide the analog sensor signals to the control unit 114. The control unit 114 can include an analog-to-digital converter (ADC) that converts the analog sensor signals into digital sensor data. The digital sensor data can be utilized by the control unit 114 to predict blood pressure. Alternatively, the inertial measurement units 106 and 108 generate digital sensor data and provide the digital sensor data to the control unit 114.

In one embodiment, the control unit 114 includes digital signal processing (DSP) circuitry that can condition the sensor data generated by the ADC. The DSP may include filters, feature generators, or other circuitry that may condition the digital sensor data prior to the analysis of the digital sensor data to predict or estimate the blood pressure. In one embodiment, the inertial measurement units 106 and 108 can include embedded ADCs to convert the analog sensor signals to digital sensor data prior to providing the digital sensor data to the control unit 114. In one embodiment, the inertial measurement units 106 and 108 can include DSPs for conditioning the digital sensor data prior to providing the digital sensor data to the control unit 114. In one embodiment the inertial measurement units 106 and 108 can include digital signal processing blocks and a processor which can execute logic written in high-level languages (such as C/C++).

In one embodiment, the control unit 114 includes one or more display elements. The one or more display elements can include one or more LEDs. The one or more LEDs can have one or more illumination schemes that indicate whether the blood pressure monitoring device 102 is powered on, whether the blood pressure monitoring device 102 is functioning properly or improperly, whether an alignment process is ongoing, whether a battery of the control unit 114 is low, or various other types of indications.

In one embodiment, the control unit 114 can include one or more user input devices. The user input devices can include buttons, switches, keys, touch screens, or other suitable devices by which a user can input commands to the control unit 114. The input devices can be utilized to determine the blood pressure monitoring device 102 on or off, to select an operating mode of the blood pressure monitoring device, or to perform other functions.

In some embodiments, the control unit 104 includes a microcontroller, a microprocessor, a logic circuit, or other types of processing circuitry.

The system 100 includes the electronic device 104. The electronic device 104 is an electronic device that is wirelessly paired or otherwise coupled to blood pressure monitoring device 102. In an exemplary embodiment described herein, the electronic device 104 is a mobile phone. However, in one embodiment, the electronic device 104 includes a smartwatch, a tablet, smart glasses, a laptop computer, a desktop computer, or another type of computing device that can communicate wirelessly with the blood pressure monitoring device 102.

The electronic device 104 includes a sensor processing module 118. The sensor processing module 118 includes a combination of hardware circuitry and software instructions that enable the electronic device 104 to process sensor data from the blood pressure monitoring device 102 and to implement a method for assisting the user to properly align the blood pressure monitoring device 102.

In some embodiments, the electronic device 104 includes processing resources. The processing resources include one or more processors, microcontrollers, logic circuits, or other processing resources. In some embodiments, the electronic device 104 includes memory resources. The memory resources configured to store software instructions another type of data. The one or more processors can execute software instructions or otherwise utilize data stored in the memories. In one embodiment, the electronic device 104 includes communication resources. The communication resources enable communication between components of the electronic device 104 and between the electronic device 104 and components external to the electronic device software, such as the blood pressure monitoring device 102.

In one embodiment, the sensor processing module 118 utilizes processing resources, memory resources, and communication resources of the electronic device 104 to process sensor data from the blood pressure monitoring device one active, to implement an alignment method of the blood pressure monitoring device 102, and to generate blood pressure measurements based on the sensor data.

In one embodiment, the sensor processing module implements or includes a blood pressure monitoring application. The application includes a software application executed by the control device 104 two facilitate the alignment process and generation of blood pressure measurements. For example, in one embodiment the electronic device 104 is a smart phone. The user can select an icon corresponding to the application. The application can then guide the user through the blood pressure monitoring device alignment process and output blood pressure measurements. The application is executed by the processing and memory resources of the electronic device 104.

In one embodiment, the electronic device 104 includes a wireless transceiver 120. The wireless transceiver 120 is part of the communication resources guide previously. The wireless transceiver 120 receives sensor data and other data from the wireless transceiver 116 of the control unit 114 of the blood pressure monitoring device 102. The wireless transceiver 120 can operate in accordance with wireless communication protocols, including Bluetooth, Wi-Fi, or other suitable wireless communication protocols or standards.

In one embodiment, the electronic device 104 includes a display 122. The display 120 can display text, images, or videos. The display 122 can output instructions to the user related to the alignment process of the blood pressure monitoring device 102. The instructions can include textual instructions, still images, or videos indicating adjustments for the user to make to the adjuster mechanism 112. The display 122 can display an estimated blood pressure, including both systolic blood pressure and diastolic blood pressure. The digital display can display indications of proper function, abnormal function, low power, unsafe blood pressure levels, or other types of information. The digital display can include a liquid crystal display (LCD) or other types of displays.

The electronic device 104 includes a speaker 124. The speaker 124 can output audible instructions to the user during an alignment process of the blood pressure monitoring device 102. The instructions can include telling the user to adjust the adjuster mechanism 112 left, right, up, down, clockwise, or anticlockwise. The speaker 124 can indicate to the user that the blood pressure monitoring device 102 is properly aligned. The speaker 124 can output the blood pressure measurements to the user.

In one embodiment, the system 100 operates in the following manner. The user places the blood pressure monitoring device 102 on the skin at a selected location. In one example, the user attempts to place the blood pressure monitoring device such that the sensor housing one interior is aligned over the carotid artery on the right side of the user's neck. The user then turns on the blood pressure monitoring device by pressing a power button or in another suitable way. The user accesses the electronic device 104 and activates a blood pressure monitoring application associated with the blood pressure monitoring device 102. The user indicates to the application (e.g., via touchscreen or voice command) that the user would like to make a blood pressure measurement. The application then initiates the alignment process of the blood pressure monitoring device 102.

During the alignment process, inertial measurement units 106 and 108 generate initial sensor data and transmits the initial sensor data to the electronic device 104. The application (implement other executed by the sensor processing module 118) then processes the sensor data extractor generate one or more parameters based on the initial sensor data. The application then instructs the user, visually or audibly, to make an initial manipulation of the adjuster mechanism 112. The initial manipulation can include translating the adjuster mechanism 112 one step to the left, one step to the right, one step up from one step down, one step clockwise, or one step anticlockwise.

After the initial manipulation, the inertial measurement units 106 and 108 provide a current set of sensor data with the electronic device 104. The application analyzes the set of sensor data and extracts or generates current values of the one or more parameters on the current set of sensor data. If current values of the one or more parameters indicate worsening alignment compared to the initial sensor data, then the electronic device outputs instructions to the user to revert the previous adjustment and to make a new adjustment. If the current values of the one or more parameters indicate improving alignment, the application outputs instructions for the user to make a further adjustment in the same direction (e.g., another step left, right, up, down, CW, or AW).

After the next manipulation, the inertial measurement units 106 and 108 provide a current set of sensor data with the electronic device 104. The application analyzes the set of sensor data and extracts or generates current values of the one or more parameters on the current set of sensor data. If the current values of the one or more parameters indicate worsening alignment compared to the previous values, then the electronic device outputs instructions to the user to revert the previous adjustment and to either make a new adjustment or keep the alignment at the previous stage if the application determines that the previous location is optimally or acceptably aligned. If the current values of the one or more parameters indicate improving alignment, the application again outputs instructions for the user to make another adjustment in the same direction (e.g., another step left, right, up, down, CW, or AW).

The alignment process continues in iterations until an optimal or acceptable alignment position has been found. In one embodiment, the electronic device 104 determines that the optimal or acceptable alignment position has been found when all further adjustments from that position result in worsening alignment. In a simple illustrative example, the initial adjustment is a single step to the left and results in an improved set of parameters in the sensor data. A subsequent adjustment further to the left results in worsening alignment as indicated by worsening sensor data parameter values. Reversion to the previous location and a CW adjustment results in worsening alignment. Reversion to the previous location and an AW adjustment results in worsening alignment. Reversion and adjustment up and down likewise resulted in worsening alignment. The application determines that the location of the first adjustment is the optimal or acceptable alignment position or orientation. Other types of adjustment processes can be utilized in accordance with principles of the present disclosure without departing from the scope of the present disclosure.

The application indicates to the user that the blood pressure monitoring device 102 is aligned. The inertial measurement units 106 and 108 output sensor data from the aligned position. The application generates a blood pressure measurement for the user based on the sensor data from the aligned location. The application outputs the blood pressure measurement data by either the display 122 or the speaker 124.

In one embodiment, the sensor processing module includes an analysis model. The analysis model receives the digital sensor data, processes the digital sensor data, and generates a blood pressure prediction or estimation based on the digital sensor data from the inertial measurement units 106 and 108 from the aligned position.

In one embodiment, the analysis model is trained with a multipart machine learning process. An initial or general machine learning process trains the analysis model with an initial training set that is generated from sensor data and blood pressure measurements of a plurality of individuals that utilized blood pressure monitoring devices similar or identical to the blood pressure monitoring device 102.

The initial training set data may be gathered in the following manner: Each of a plurality of individuals may wear a blood pressure monitoring device including a first and a second inertial measurement unit similar or identical to the inertial measurement units 106 and 108. The inertial measurement units generates sensor data for selected periods of time while the individuals wear the blood pressure monitoring devices. The individuals also utilize a control blood pressure monitor that measures the blood pressure of the individuals during the selected periods of time. The blood pressure measurements are labeled for the training set. Each set of sensor data is matched with a blood pressure measurement. The machine learning process trains the analysis model to accurately predict the blood pressure based on the sets of sensor data. When the predicted blood pressures match the measured blood pressures of the labels within a selected error tolerance the initial machine learning process is complete. This corresponds to pre-training the analysis model.

When the blood pressure monitoring device 102 is provided to the user, the pre-trained analysis model is preloaded in the sensor processing module 118. The pre-trained analysis model may be utilized to predict the blood pressure of the individual based on sensor data from the inertial measurement units 106 and 108. At this point, the blood pressure predictions made by the analysis model may be fairly accurate. However, improved accuracy can be achieved by performing a second, individualized machine learning process.

The second machine learning process is an individualized machine learning process for the user of the blood pressure monitoring device 102. After the user has obtained the blood pressure monitoring device 102, the user can utilize the application to enter an individualized machine learning process. At this point, the user may be prompted to put on both the blood pressure monitoring device 102 and a control blood pressure monitor.

During the individualized machine learning process, the sensor processing module records sensor data from the inertial measurement units 106 and 108 over a select period of time. The recorded sensor data makes up part of an individualized training set. The control blood pressure monitor also generates blood pressure measurements during the selected period of time. The blood pressure measurements correspond to labeled data for the individualized training set.

During the machine learning process, the analysis model is retrained utilizing the individualized training set. The analysis model is trained with the individualized training set to work within a threshold error tolerance. In this way, the analysis model is trained to predict or estimate the blood pressure of the individual with high accuracy based on sensor data from the inertial measurement units 106 and 108.

In one embodiment, the analysis model 152 implements a neural network. The neural network includes a plurality of neural layers. The initial or general machine learning process trains each of the layers of the neural network. Training the layers of the neural networks can include identifying weighting values for functions associated with edges or connections between neurons of different neural layers.

In one embodiment, during the second or individualized machine learning process retrains one or more of the layers of the neural network. During the second or individualized machine learning process, one or more other layers of the neural network are held fixed such that functions on weighting values associated with the fixed layers are not retrained during the second or individualized machine learning process. Retraining only a portion of the neural network results in a neural network that has the benefits of the general machine learning process and the individualized machine learning process. After the individualized machine learning process, the analysis model can make blood pressure estimations or predictions with great accuracy for the individual based on the sensor data from the inertial measurement units 106 and 108.

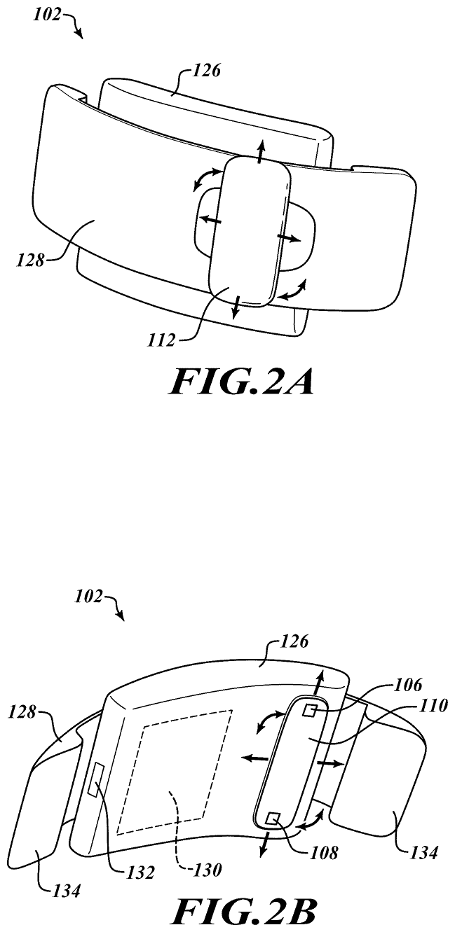

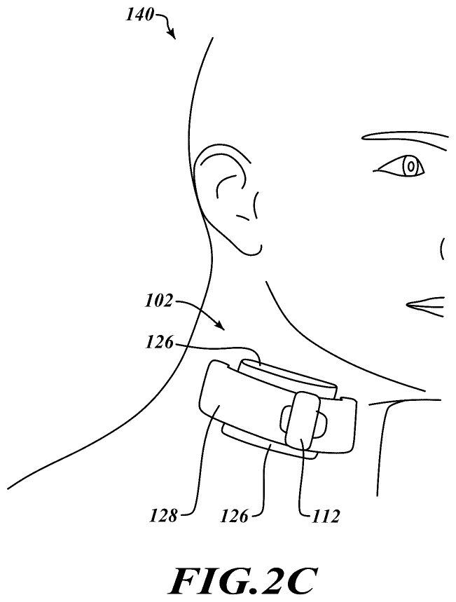

FIGS. 2A-2C are illustrations of a blood pressure monitoring device 102, according to one embodiment. The blood pressure monitoring device 102 of FIGS. 2A-2C is one example of a blood pressure monitoring device 102 of FIG. 1.

With reference to FIGS. 2A and 2B, the blood pressure monitoring device 102 includes a main housing 126 and a strap 128. The adjustment mechanism is mechanically coupled to the sensor housing 110 through the main housing 126. The adjustment mechanism 112 is adjacent to an opening in the strap 128 that enables the adjustment mechanism 112 to translate and rotate relative to the strap 128, as described previously. Though not apparent in the views of FIGS. 2A-2C, the main housing 126 includes one or more groups, slots, or other coping mechanisms that enable the adjuster mechanism 112 to couple to the interior housing 110 and to enable translation and rotation in discrete steps.

In one embodiment, each discrete translation step is 1 mm (left, right, up, or down). In one embodiment, each rotation step is point 0.5°. Other values of translation and rotation steps can be utilized without departing from the scope of the present disclosure.

In one embodiment, the strap 128 includes adhesive portions 134 at the ends of the strap 128. The adhesive ends 134 include a material that lightly adheres to the skin of the user and that can then be removed from the skin of the user without damaging the skin of the user.

The blood pressure monitoring device 102 includes a main housing 126 and a sensor housing 110. The sensor housing is mechanically coupled to the adjustment mechanism 112 such that moving the adjustment mechanism also moves the sensor housing 110 relative to the main housing 126. As set forth previously, the sensor housing 110 houses the inertial measurement units 106 and 108.

The main housing 126 includes circuitry 130, represented in dashed lines in FIG. 2B. The circuitry 130 includes the control circuitry 114 shown and described in relation to FIG. 1. Furthermore, the circuitry 130 can include one or more batteries, memories or other circuitry. The circuitry 130 receives sensor data from the inertial measurement units 106 and 108 and transmits the sensor data to the electronic device 104. In some embodiments, the control circuitry 130 can condition, format, or process the sensor data prior to sending the sensor data to the electronic device. In some embodiments, the circuitry 130 extracts features or generates parameters based on the sensor data and sends them to the electronic device 104.

The main housing includes a port 132. The port 132 is configured to receive a charging cord to charge a battery of the blood pressure monitoring device. The port 132 is also configured to receive data and provide the data to the circuitry 130, such as firmware updates or other data. The port 132 is also configured to output data from the circuitry 130.

FIG. 2C illustrates the blood pressure monitoring patch 102 positioned on the skin of a user 140. In FIG. 2C, the blood pressure monitoring device 102 is positioned on the right side of the user's neck over the carotid artery. However, the blood pressure monitoring device 102 can be positioned on other locations on a user without departing from the scope of the present disclosure.

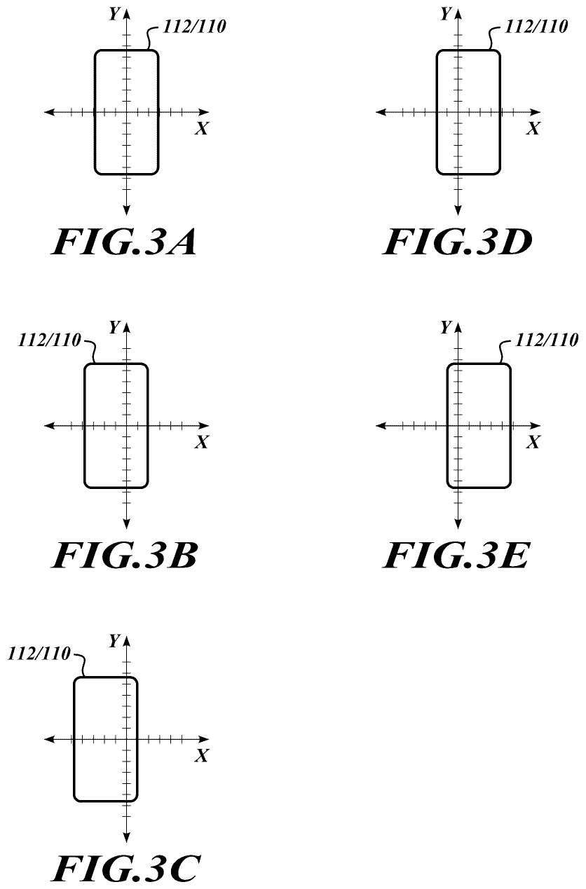

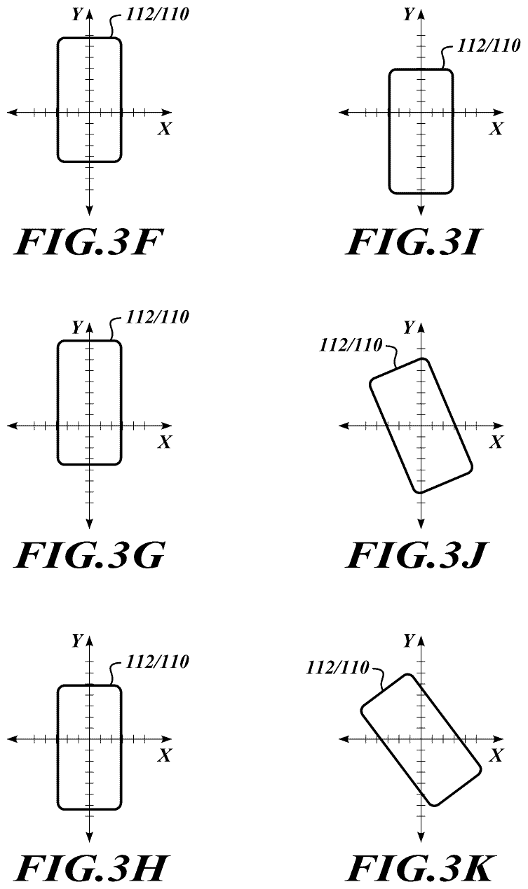

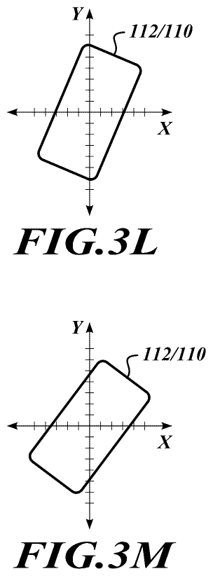

FIGS. 3A-3M illustrate the orientation of the sensor housing 110/adjustment mechanism 112. In FIG. 3A, the sensor housing 110/adjustment mechanism 112 is at a default or central position. In FIGS. 3B and 3C, the sensor housing 110/adjustment mechanism 112 has been translated once step (3B) and then two steps (3C) to the left from the default position. In FIGS. 3D and 3E, the sensor housing 110/adjustment mechanism 112 has been translated once step (3D) and then two steps (3E) to the right from the default position.

In FIGS. 3F and 3G, the sensor housing 110/adjustment mechanism 112 has been translated one step (3F) and then two steps (3G) up from the default position. In FIGS. 3H and 3I, the sensor housing 110/adjustment mechanism 112 has been translated one step (3H) and then two steps (3I) down right from the default position.

In FIGS. 3J and 3K, the sensor housing 110/adjustment mechanism 112 has been rotated one step (3J) and then two steps (3K) AW from the default position. In FIGS. 3L and 3M, the sensor housing 110/adjustment mechanism 112 has been rotated one step (3L) and then two steps (3M) down right from the default position.

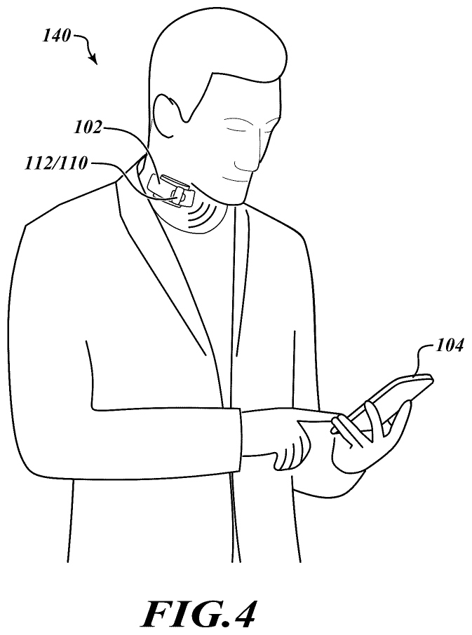

FIG. 4 illustrates a user 140 utilizing a blood pressure monitoring device 102 and an electronic device 104 during an alignment and measurement process. The blood pressure monitoring device 102 is positioned on the neck of the user over the carotid artery. The user has accessed the blood pressure monitoring device application on the electronic device (smart phone) 104. The blood pressure monitoring device outputs sensor data to the electronic device 104. The electronic device 104 processes the sensor data and guides the user in aligning the blood pressure monitoring device 102 by instructing the user to make a series of adjustments to the adjustment mechanism 112, as described previously. After the alignment process is complete, the electronic device 104 generates a blood pressure measurement based on sensor data from the aligned position.

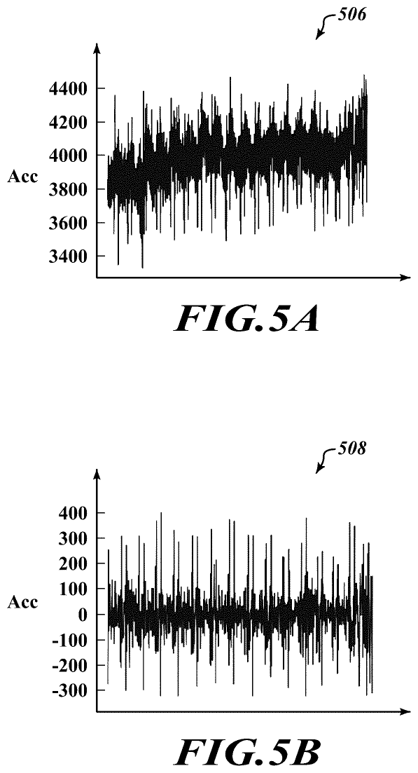

FIG. 5A is a graph 506 sensor data from the inertial measurement unit 106, in accordance with one embodiment. The graph illustrates the magnitude of acceleration signals over time at a given orientation/position on a user's neck. FIG. 5B is a graph 508 sensor data from the inertial measurement unit 108, in accordance with one embodiment. The graph illustrates the magnitude of acceleration signals over time at a given orientation/position on a user's neck. As can be seen from the graph's 506/508, the sensor from inertial measurement units 106 and 108 is very different due to misalignment over the carotid artery.

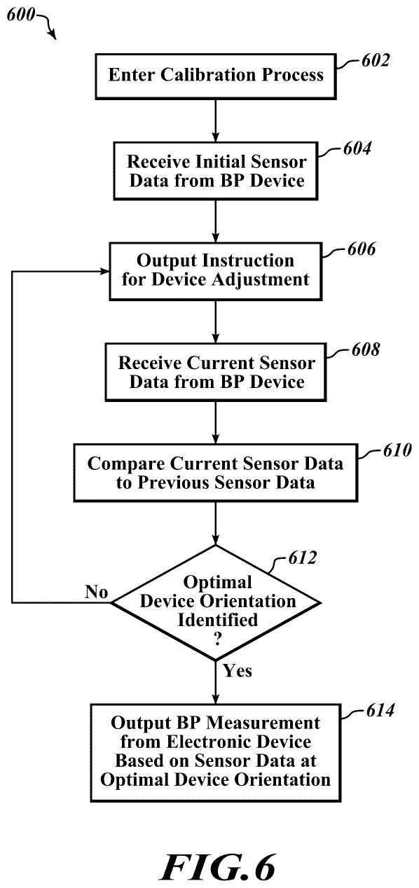

FIG. 6 is a flow diagram of a method 600 of aligning a blood pressure monitoring device 102, in accordance with one embodiment. At 602, a blood pressure monitoring device 102 and electronic device 104 enter a calibration or alignment process. In one example this corresponds to the user powering on the blood pressure monitoring device 102 and accessing the corresponding application on the electronic device 104. At 604, the electronic device 104 receives an initial set of sensor data from the inertial measurement units 106 and 108. At 606, the electronic device 104 processes the sensor data and outputs an instruction to the user to make a particular adjustment with the adjustment mechanism 112. At 608, the electronic device 104 receives a current set of sensor data from the inertial measurement units 106 and 108 from the current orientation. At 610, the electronic device 104 compares the most recent set of sensor data to previous sensor data. At 612, the electronic device 104 checks to see whether the optimal alignment orientation has been identified. In one embodiment, the optimal alignment orientation is identified when adjustments from that position does not yield improved parameters from the sensor data. At 612, if the optimal orientation has not been identified, the process returns to 606 and a new adjustment is instructed. At 612, if the optimal orientation has been identified, then the process proceeds to 614. At 614, the electronic device 104 generates a blood pressure measurement based on the sensor data at the aligned position. The electronic device 104 outputs the blood pressure measurement to the user.

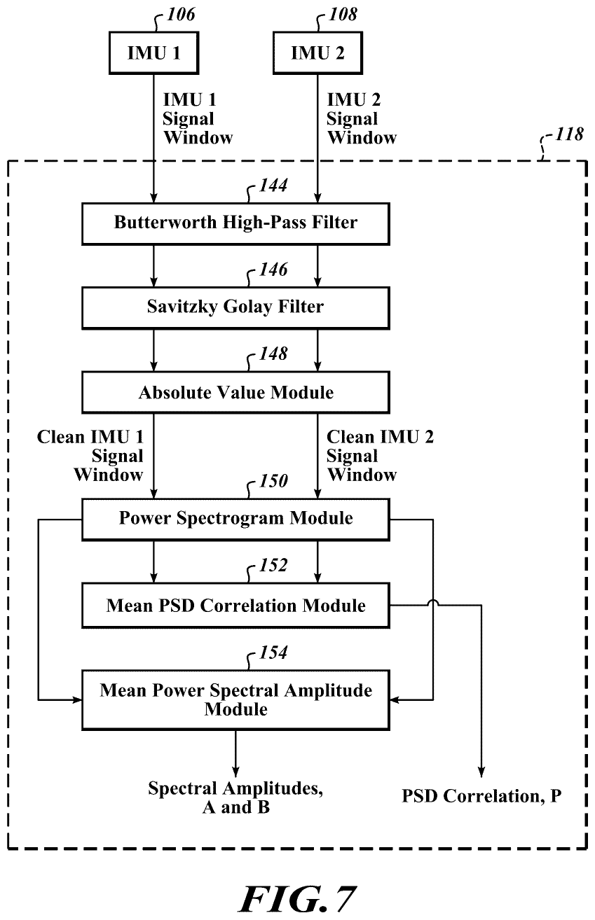

FIG. 7 is a block diagram illustrating generation of selected parameters by the electronic device 104 from the sensor data of inertial measurement units 106 and 108, in accordance with one embodiment. FIG. 7 illustrates the inertial measurement units 106 and 108 providing sensor data to the control circuitry 118 of the electronic device 104. More particularly, the blood pressure monitoring device 102 transmits a set of sensor data from each of the inertial measurement units 106 and 108 in a particular signal window. A signal window includes a set of samples of the sensor data for the corresponding inertial measurement unit. In one embodiment, the sensor data includes acceleration data for each of three axes of the three axis inertial measurement units 106 and 108.

The control circuitry 118 includes a Butterworth high pass filter 144. The Butterworth high pass filter 144 receives the sensor data from the inertial measurement units 106 and 108 and filters the data with a high pass filter. In one embodiment, the Butterworth high pass filter has an order of 2 and a cutoff frequency of 2 Hz, though other orders and cutoff frequencies can be utilized without departing from the scope of the present disclosure.

The sensor processing unit 118 includes a Savitzky-Golay filter 146 downstream from the Butterworth high pass filter 144. The Savitzky-Golay filter 146 receives the sensor data from the Butterworth high pass filter 144 and further filters the sensor data. In one embodiment, the Savitzky-Golay filter 146 has a filter window of fs/2.5 and an order of six, though other windows and orders can be utilized without departing from the scope of the present disclosure.

The sensor processing unit 118 includes an absolute value module 148 downstream from the Savitzky-Golay filter 146, in accordance with one embodiment. The absolute value module 148 receives the filtered sensor data from the Savitzky-Golay filter 146 and extracts the absolute values of the sensor data.

The sensor processing unit 118 includes a power spectrogram module 150 downstream from the absolute value module 148, in accordance with one embodiment. The power spectrogram module 150 receives the clean sensor data of the inertial measurement units 106 and 108 from the absolute value module 148. The power spectrogram module 150 generates power spectrogram data from the sensor data. The sensor data is a time domain signal. The power spectrogram data, includes, for each of a plurality of frequency components present in the time domain signal, a corresponding proportional to the amplitude squared.

The sensor processing unit 118 includes a main power spectral density (PSD) correlation module 152 downstream from the power spectrogram 150, in accordance with one embodiment. The mean PSD correlation module 152 receives the power spectrogram data from the power spectrogram module 150.

The mean PSD correlation module 152 generates a PSD for the sensor data from each inertial measurement unit. The PSD of a signal describes how the power of a signal is distributed across different frequency components. The mean PSD correlation module then generates the mean PSD for the sensor data of each inertial measurement unit, in the following manner:

$$Mean PSDx = \frac{1}{{f}_{2} - {f}_{1}} \int {{f}_{1}}^{{f}_{2}} {S}_{x} ( f ) , df ,$$

$$Mean PSDy = \frac{1}{{f}_{2} - {f}_{1}} \int {{f}_{1}}^{{f}_{2}} {S}_{y} ( f ) , df$$

where Mean PSDx is the mean PSD of the inertial measurement unit 106 (accordingly, here, x does not refer to an inertial measurement unit axis but to the inertial measurement unit 106) and Mean PSDy is the mean PSD of the inertial measurement unit 108 (accordingly, here, y does not refer to an inertial measurement unit axis but to the inertial measurement unit 108). S(f) is calculated in the following manner:

S_{A}(f)=|A(f)|²,
where A(f) is calculated from the sensor data a(t) by using a discrete Fourier transform (DFT) in the following manner:

$$A ( f ) = {\int }_{- ∞}^{∞} a ( t ) {e}^{- j 2 π ft} , dt .$$

The mean PSD correlation P corresponds to the covariance between Mean PSDx and Mean PSDy, divided by the product of the standard deviations (6) of the Mean PSDx and Mean PSDy, as set forth in the following formal:

$$P = \frac{Cov ( Mean {PSD}_{x} , Mean {PSD}_{y} )}{σ Mean PSDx σ Mean {PSD}_{y}} .$$

The mean PSD correlation P is a single value and corresponds to a parameter used by the sensor processing unit 118 during the alignment process, as will be set forth in more detail below.

Returning to FIG. 7, the sensor processing unit 118 also includes a mean power spectral amplitude module 154, in accordance with one embodiment. The main power spectral amplitude module 154 is downstream from the power spectrogram module 150 and receives the power spectrogram data from the power spectrogram module 150. The mean power spectral amplitude module 154 generates mean spectral amplitudes A and B. A is the average of the n highest coefficients of the PSD of the sensor data for the inertial measurement unit 106. B is the average of the n highest coefficients of the PSD of the sensor data for the inertial measurement unit 108.

In one embodiment, A and B are calculated by first computing the discrete Fourier transform (DFT) for the sensor data a(t) from each inertial measurement unit, resulting in N values of A(f), using the formula set forth above. Next, the PSD coefficients S(f) are calculated for the N values of A(f), as set forth above. The PSD coefficients are then sorted in descending order. The n top values of the coefficients are selected for the sensor data from each inertial measurement unit 106 and 108. A is then generated as the average of the n highest PSD coefficients associated with the inertial measurement unit 106. B is then generated as the average of the n highest PSD coefficients associated with the inertial measurement unit 108. A and B are parameters utilized by the sensor processing unit 118 during the alignment process, as will be set forth in more detail below.

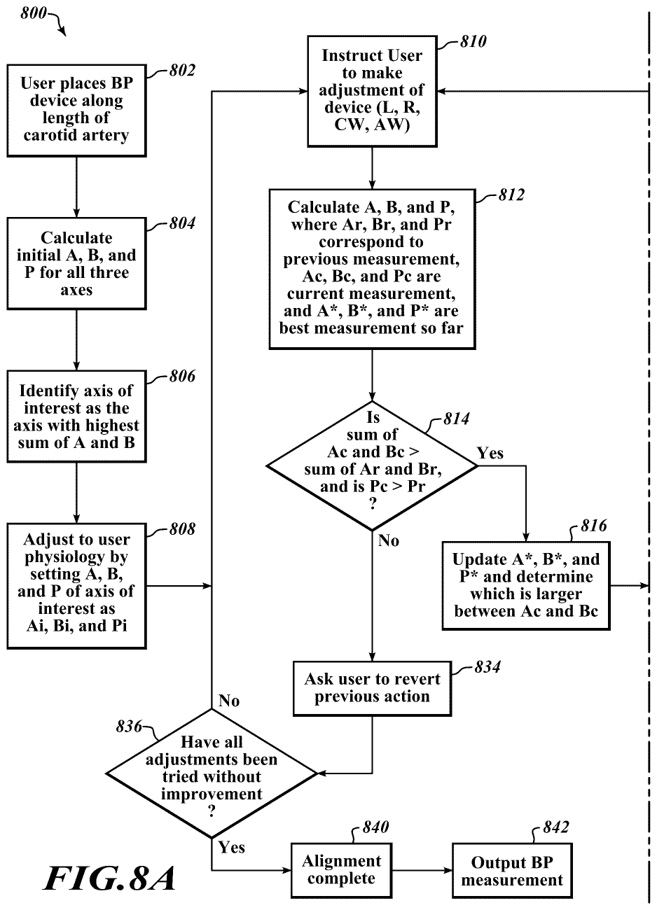

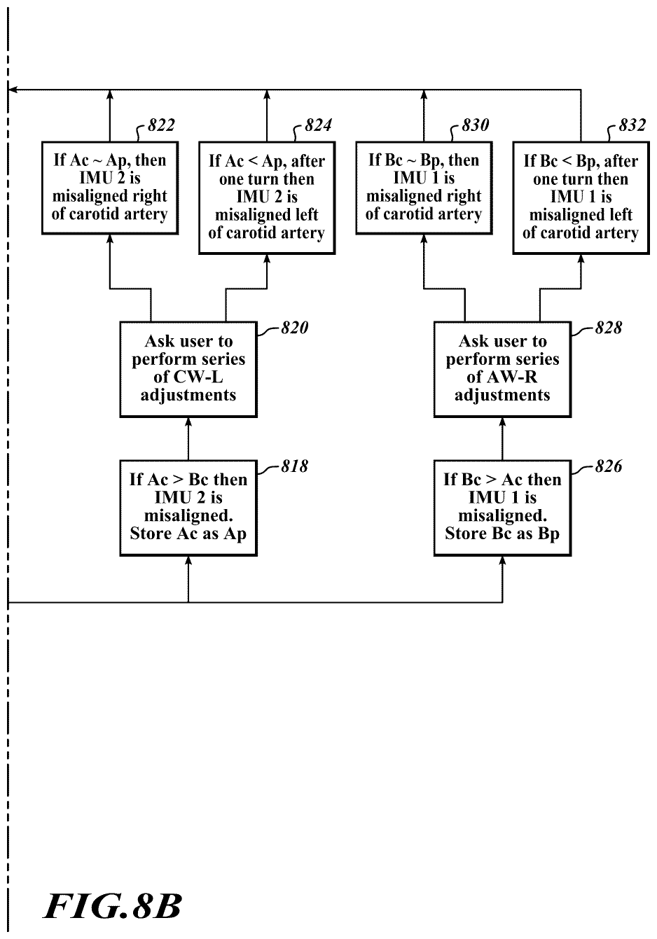

FIGS. 8A and 8B collectively illustrate a flow diagram of a process 800 for aligning a blood pressure monitoring device 102, in accordance with one embodiment. With reference first to FIG. 8A, at 802 the user places the blood pressure monitoring device 102 on the skin such, attempting to place the sensor housing 110 over the carotid artery on the right side of the neck. At 804, the sensor processing unit 118 of the electronic device 104 receives sensor data from the inertial measurement units 106 and 108. The sensor processing unit 118 calculates initial values of A, B, and P for all three axes of the inertial measurement units 106 and 108. At 806, the sensor processing unit 118 of the electronic device 104 identifies the axis of interest as the axis with the highest sum of A and B.

In one embodiment, the Z axis is highly likely to have the highest sum of A and B. Accordingly, in one embodiment, the Z axis is selected as the axis of interest by default.

At 808, the sensor processing unit 118 adjusts to the user physiology by setting A, B, and P of the axis of interest as Ai, Bi, and Pi. The blood pressure signals captured by the blood pressure monitoring device 102 depends on a variety of user physiological factors such as arterial thickness, fat tissue thickness, facial hair thickness, etc. During the axis of interest calculation (806), Aᵢ, Bᵢ, and Pᵢ also corresponds to personalized metrics for the user, such that when adjusting the device, the new values of A, B and P are centered around Aᵢ, Bᵢ, and Pᵢ, in one embodiment. From here, the sensor processing unit 118 considers the sensor data only from the axis of interest.

At 810, the electronic device 104 instructs the user to make an adjustment of the adjustment mechanism 112, thereby adjusting the sensor housing 110 and the inertial measurement units 106 and 108 and the corresponding manner. The initial adjustment can include a translation to the left or right or rotation CW or AW.

At 812, the sensor processing unit 118 calculates A, B, and P. Ar, Br, and Pr correspond to the previous measurement, Ac, Bc, and Pc correspond to the current measurement, and A*, B*, and C* correspond to the best measurement so far.

At 814, the sensor processing unit determines whether the sum of Ac and Bc is greater than the sum of Ar and Br and if Pc is greater than Pr. If both conditions are true, then the process proceeds to 816. If either condition is not true, then the process proceeds to 834.

At 834, the electronic device 104 instructs the user to revert the previous action. In other words, the electronic device 104 instructs the user to revert the adjustment mechanism 112 to the position immediately prior to the most recent adjustment. If the most recent adjustment was a translation to the right by one step, then the reversion is a translation to the left by one step. If the most recent adjustment was a CW rotation by one step, then the reversion is an AW rotation by one step, and so forth.

At 836, the electronic device 104 determines whether all adjustments have been tried without improvement to A*, B*, and P*. If all adjustments have been tried without improvement, then at 840 alignment is complete. The electronic device then processes the sensor data from the aligned position in measurement or estimation of the blood pressure of the user at 842. The electronic device 104 outputs the blood pressure measurement to the user. At 836, if all adjustments have not been tried without improvement, the process returns to 810 at which point the electronic device 104 instructs the user to make a new adjustment.

At 816, the sensor processing unit updates A*, B*, and P* with the values Ac, Bc, and Pc based on the comparisons performed at 814. Furthermore, the sensor processing unit 118 also determines which is larger between Ac and Bc (now A* and B*). From 816, the process proceeds to 818 or 826 as shown in FIG. 8B.

With reference to FIG. 8B, if Ac is greater than Bc, then at 818 the sensor processing unit 118 determines that the inertial measurement unit 108 is misaligned and stores Ac as Ap. At 820, the electronic device asked the user to perform a series of CW or L adjustments. After the first adjustment, the sensor processing unit 118 processes the sensor data to generate new Ac, Bc, and Pc. If Ac is less than Ap after the first CW/L adjustment, then at 824 the inertial measurement unit 108 is misaligned left of the carotid artery and the next adjustment is AW or R, which instruction is given at 810. If Ac~Ap, then the inertial measurement unit 108 is misaligned right of the carotid artery at 822 and the process returns to 810 and a new adjustment instructions is output to the user.

At 816, if Bc is greater than Ac, then at 826 the sensor processing unit 118 determines that the inertial measurement unit 106 is misaligned and stores Bc as Bp. At 828, the electronic device asked the user to perform a series of AW or R adjustments. After the first adjustment, the sensor processing unit 118 processes the sensor data to generate new Ac, Bc, and Pc. If Bc is less than Bp after the first AW/R adjustment, then at 832 the inertial measurement unit 106 is misaligned left of the carotid artery and the next adjustment is CW or L, which instruction is given at 810. If Bc~ Bp, then the inertial measurement unit 108 is misaligned right of the carotid artery at 830 and the process returns to 810 and a new adjustment instructions is output to the user.

The process continues until at 836 all adjustments have been tried without improvement to A*, B*, and P*. At which point alignment is complete at 840 and a blood pressure measurement is output at 842, as described previously. FIGS. 8A and 8B illustrate one possible process for aligning the blood pressure monitoring device 102, in accordance with one embodiment. Those of skill in the art will recognize, in light of the teachings of the present disclosure, that other processes and parameters can be utilized to perform an alignment process of a blood pressure monitoring device 102. Also such other processes and parameters fall within the scope of the present disclosure.

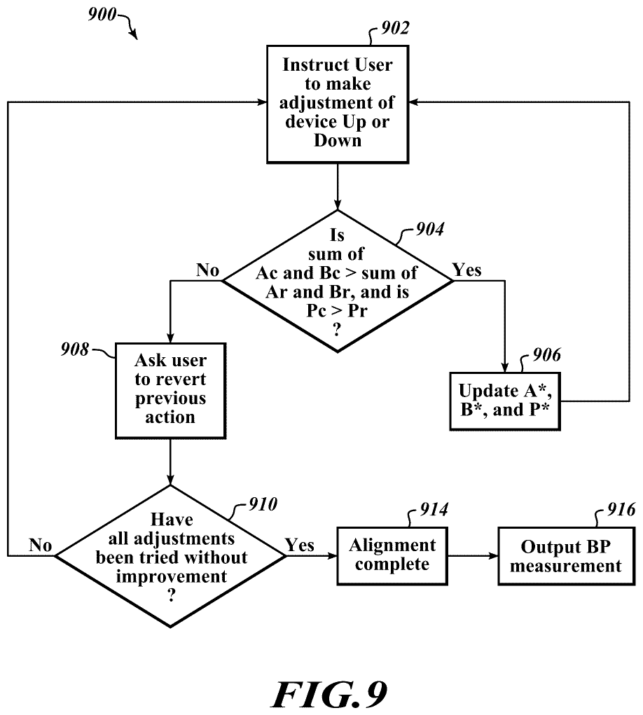

FIG. 9 illustrates is a flow diagram of a process 900 that can be utilized to supplement the process described in relation to FIGS. 8A and 8B, in accordance with one embodiment. FIGS. 8A and 8B do not utilize up-and-down translations. This is because L/R or CW/AW rotations are more likely to result in a change from misalignment to alignment. However, once the inertial measurement units 106 and 108 are aligned over the carotid artery, it can be more beneficial to make translations up or down to slightly enhance the strength of the sensor data. Accordingly, in one embodiment, the process 900 is utilized after step 836 of the process 800.

At 902, the electronic device 104 instructs the user to make an adjustment of the patch up or down, in accordance with one embodiment. At 904, the sensor processing unit determines whether the sum of Ac and Bc is greater than the sum of Ar and Br, and if Pc is greater than Pr. If no, then the process proceeds to 908. If yes, then the process proceeds to 906. At 906, A*, B*, and P* are updated and the process returns to 902.

At 908, the electronic device 104 asked the user to revert the previous action. At 910, sensor processing unit 118 determines whether all up/down adjustments have been tried without improvement. If yes, then alignment is completed 914 and the sensor processing unit generates a blood pressure measurements in the electronic device outputs the blood pressure measurements at 916. If no, the process returns to 902.

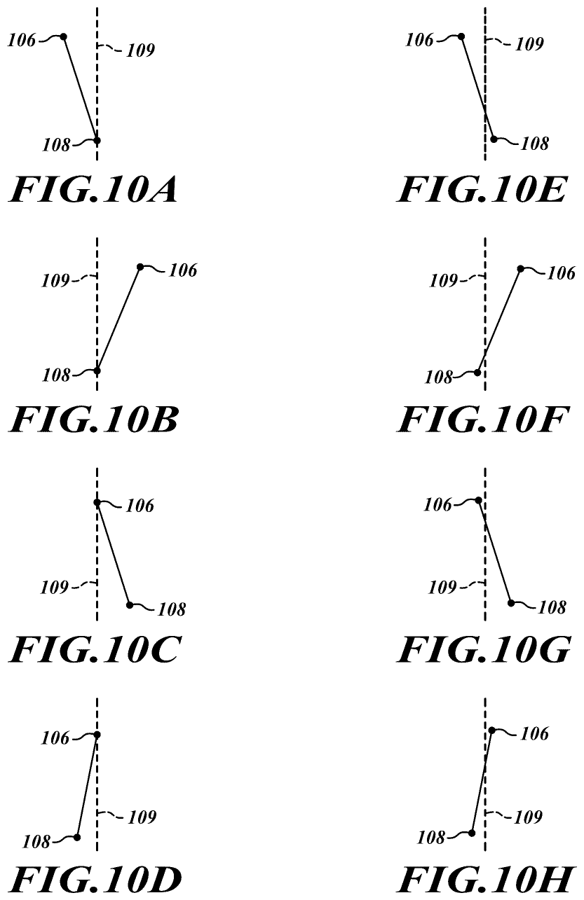

FIGS. 10A-10H are illustrations of various orientations of the inertial measurement units 106 and 108 relative to the carotid artery 109, in accordance with one embodiment.

In FIG. 10A, the inertial measurement unit 108 is aligned over the carotid artery 109, while the inertial measurement unit 106 is misaligned to the left of the carotid artery 109. In this case, a combination of one or more CW rotations and one or more translations to the right may result in both the inertial measurement unit 106 and the inertial measurement unit 108 being positioned directly over the carotid artery 109.

In FIG. 10B, the inertial measurement unit 108 is aligned over the carotid artery 109, while the inertial measurement unit 106 is misaligned to the right of the carotid artery 109. In this case, a combination of one or more AW rotations and one or more translations to the left may result in both the inertial measurement unit 106 and the inertial measurement unit 108 being positioned directly over the carotid artery 109.

In FIG. 10C, the inertial measurement unit 106 is aligned over the carotid artery 109, while the inertial measurement unit 108 is misaligned to the right of the carotid artery 109. In this case, a combination of one or more CW rotations and one or more translations to the left may result in both the inertial measurement unit 106 and the inertial measurement unit 108 being positioned directly over the carotid artery 109.

In FIG. 10D, the inertial measurement unit 106 is aligned over the carotid artery 109, while the inertial measurement unit 108 is misaligned to the left of the carotid artery 109. In this case, a combination of one or more AW rotations and one or more translations to the right may result in both the inertial measurement unit 106 and the inertial measurement unit 108 being positioned directly over the carotid artery 109.

In FIG. 10E, the inertial measurement unit 108 is misaligned to the right of the carotid artery 109, while the inertial measurement unit 106 is misaligned to the left of the carotid artery 109. In this case, one or more CW rotations, or a combination of one or more CW rotations and one or more translations to the right may result in both the inertial measurement unit 106 and the inertial measurement unit 108 being positioned directly over the carotid artery 109.

In FIG. 10F, the inertial measurement unit 108 is misaligned to the left of the carotid artery 109, while the inertial measurement unit 106 is misaligned to the right of the carotid artery 109. In this case, one or more AW rotations, or a combination of one or more AW rotations and one or more translations to the left may result in both the inertial measurement unit 106 and the inertial measurement unit 108 being positioned directly over the carotid artery 109.

In FIG. 10G, the inertial measurement unit 106 is misaligned to the left of the carotid artery 109, while the inertial measurement unit 108 is misaligned to the right of the carotid artery 109. In this case, one or more CW rotations, or a combination of one or more CW rotations and one or more translations to the left may result in both the inertial measurement unit 106 and the inertial measurement unit 108 being positioned directly over the carotid artery 109.

In FIG. 10H, the inertial measurement unit 108 is misaligned to the left of the carotid artery 109, while the inertial measurement unit 106 is misaligned to the right of the carotid artery 109. In this case, one or more AW rotations may result in both the inertial measurement unit 106 and the inertial measurement unit 108 being positioned directly over the carotid artery 109.

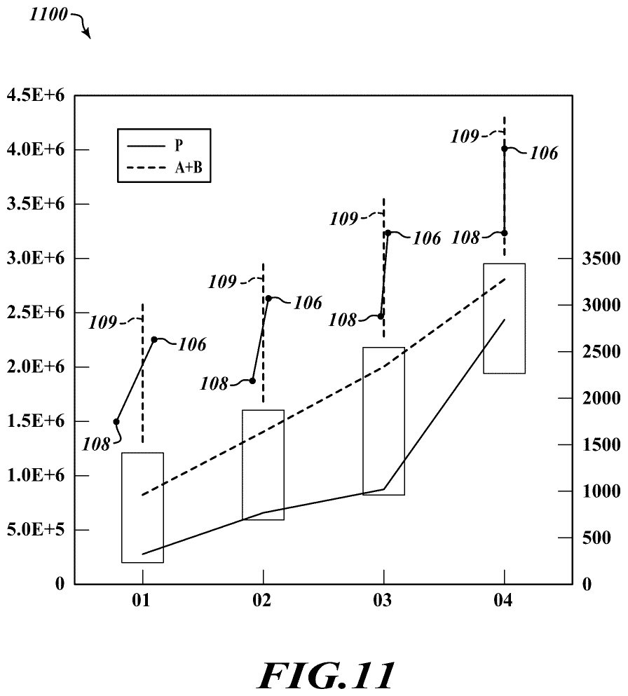

FIG. 11 is a graph 1100 illustrating A+B (dashed line) and P (continuous line) at multiple stage of an alignment process of a blood pressure monitoring device 102, in accordance with one embodiment. The x-axis corresponds to time and includes orientations 01-04, with 01 being an initial misaligned position and 04 being a final aligned position. The Y-axis on the left side of the graph corresponds to P. The Y axis on the right side of the graph corresponds to A+B. P is a unitless quantity. The units of A+B is X²/Hz, where X is the base unit of measurements. For accelerometers, the base unit of measurements is g, so A+B has unit: g²/Hz.

At position 01, the inertial measurement unit 106 is misaligned to the right of the carotid artery 109 and the inertial measurement unit 108 is misaligned to the left of the carotid artery 109. At position 02, one or more AW rotations have been performed and both the inertial measurement units 106 and 108 are closer to alignment over the carotid artery 109. At position 03, one or more translations to the right have been performed in both the inertial measurement units 106 and 108 are closer to alignment over the carotid artery 109. At position 04, one or more AW rotations have been performed and the inertial measurement units 106 and one are both aligned directly over the carotid artery 109.

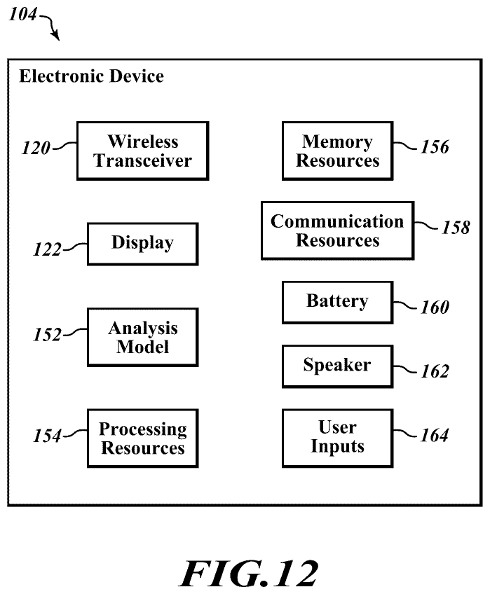

FIG. 12 is a block diagram of an electronic device 104, in accordance with some embodiments. The electronic device 104 of FIG. 12 is one example of an electronic device 104 of FIG. 1. The electronic device 104 includes a wireless transceiver 120. The wireless transceiver 120 enables the electronic device 104 to communicate with a blood pressure monitoring device 102, in accordance with one embodiment.

The electronic device 104 includes a display 122. The display 122 enables the electronic device images and video in order to provide instructions to the user during an alignment process of the blood pressure monitoring device 102. The display 122 can also display a blood pressure measurement after the alignment process.

The electronic device 104 includes analysis model 152, in one embodiment. As described previously, the analysis model can include a neural network or other type of model trained with a machine learning process to generate blood pressure measurements or estimations based on sensor data after an alignment process. The analysis model 152 can be trained with a multipart machine learning process as described previously.

The electronic device 104 includes processing resources 154. The processing resources can include one or more processors, microprocessors, controllers, logic circuits, or other types of processing resources 154. The processing resources 154 can be utilized to execute software instructions to assist in carrying out alignment processes and blood pressure measurement generation processes.

The electronic device 104 includes memory resources 156. The memory resources 156 can include one or more computer memories. The computer memories can store software instructions for performing an alignment process and for generating blood pressure measurements. The memory resources can also stir other types of data including sensor data and previous measurements.

In one embodiment, the electronic device 104 includes communication resources 158. The communication resources 158 can include components of the electronic device 104 and with components external to the electronic device 104. The communication resources 158 can include a wireless transceiver 120.

The electronic device 104 includes a battery 160. The battery 160 powers electronic device and can be recharged.

The electronic device 104 includes a speaker 162. In one embodiment, the speaker 162 outputs audible instructions to the user during the alignment process. In one embodiment, the speaker 162 audibly outputs the blood pressure measurement to the user.

The electronic device 104 includes user inputs 164. The user inputs 164 can include buttons, touchscreen, knobs, dials, or other types of mechanisms that enable a user to provide inputs or otherwise control the electronic device 104.

In one embodiment, the electronic device 104 includes a sensor processing unit 118 (see FIG. 1) that includes aspects of the processing resources 154, the memory resources 156, and the communication resources 158. In other words, the sensor processing unit 118 can be implemented utilizing the memory resources 156, processing resources 154, and communication resources 158 and the electronic device 104. In one embodiment, the electronic device 104 includes and executes a software application for performing the alignment process and the blood pressure measurement functions.

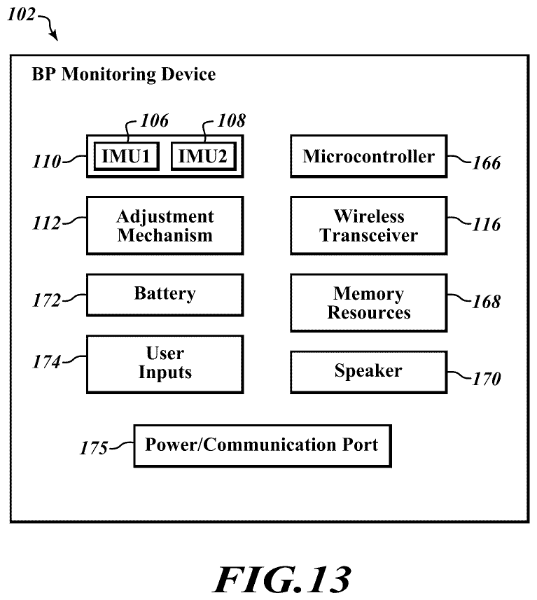

FIG. 13 is a block diagram of a blood pressure monitoring device 102, in accordance with one embodiment. The blood pressure monitoring device 102 of FIG. 13 is one example of a blood pressure monitoring device 102 of FIGS. 1-2C. The blood pressure monitoring device includes 106 and 108 coupled within the sensor housing 110, as described previously. The blood pressure monitoring device 102 includes an adjustment mechanism 112, as described previously. The blood pressure monitoring device 102 includes a wireless transceiver 116, as described previously.

In one embodiment, the blood pressure monitoring device 102 includes a microcontroller 166. The microcontroller 166 is communicatively coupled to the and the inertial measurement units 106 and 108. The microcontroller 166 controls the operation of the wireless transceiver 116 and inertial measurement units 106 and 108. The microcontroller 166 also controls the operation of other components of the blood pressure monitoring device 102.

In one embodiment, the blood pressure monitoring device 102 includes memory resources 168. The memory resources 168 can include one or more computer memories that store data related to the operation of the blood pressure monitoring device 102. The microcontroller 166 can implement instructions stored in the memory resources 168.

In one embodiment, the blood pressure monitoring device 102 includes a speaker 170. In one embodiment, the speaker 170 can output instructions to the user during an alignment process. In one embodiment, the speaker 170 can output a blood pressure measurement to the user.

In one embodiment, the blood pressure monitoring device 102 includes a battery 172. The battery 172 powers the other functions of the blood pressure monitoring device 102.

In one embodiment, the blood pressure monitoring device 102 includes user inputs 174. The user inputs 174 enable user to input commands to the blood pressure monitoring device 102. This can include turning the blood pressure monitoring device 102 on and off, and other functions.

In one embodiment, the blood pressure monitoring device 102 includes a power/communication port 175. The port 175 can receive a connecting device to facilitate battery charging or data transfer.

In one embodiment, the blood pressure monitoring device 102 implements an alignment process without the use of the electronic device 104. In particular, the microcontroller 166, in conjunction with the memory resources 168 performance of processes described in relation to the sensor processing unit 118. The blood pressure monitoring device 102 outputs alignment adjustment instructions via speaker 170 and outputs the final blood pressure measurements via the speaker 170.

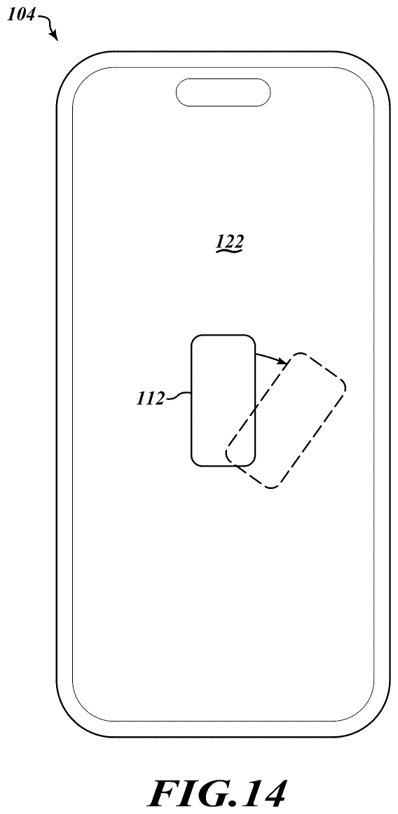

FIG. 14 is an illustration of an electronic device 104, in accordance with one embodiment. The electronic device 104 includes a display 122. During an alignment process, the display 122 can output images that provide a graphical representation of an adjustment to be made. In the example shown in FIG. 14, the display illustrates that the user should rotate the adjustment mechanism 112 in a CW direction. The electronic device 104 can also output audible instructions simultaneously with the display of the graphical instructions.

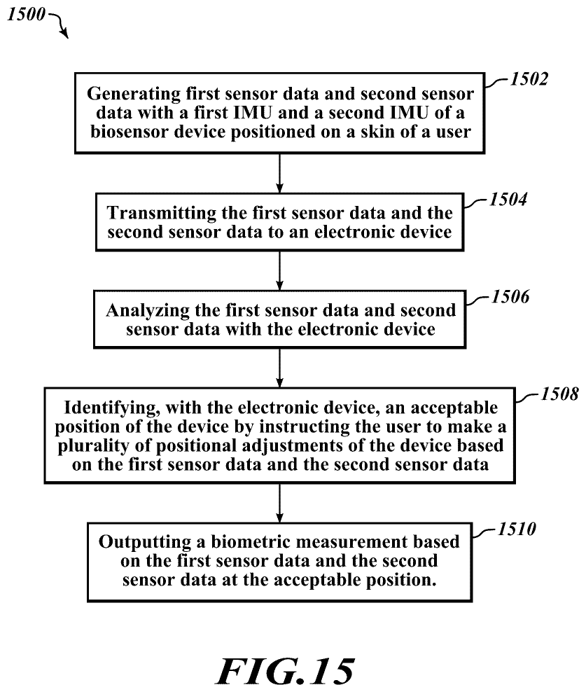

FIG. 15 is a flow diagram of a method 1500, according to one embodiment. The process 1500 can utilize systems, components, and processes described in relation to FIGS. 1-14. At 1502, the method 1500 includes generating first sensor data and second sensor data with a first inertial measurement unit and a second inertial measurement unit of a biosensor device positioned on a skin of a user. At 1504, the method 1500 includes transmitting the first sensor data and the second sensor data to an electronic device. At 1506, the method 1500 includes analyzing the first sensor data and second sensor data with the electronic device. At 1508, the method 1500 includes identifying, with the electronic device, an acceptable position of the biosensor device by instructing the user to make a plurality of positional adjustments of the patch based on the first sensor data and the second sensor data. At 1510, the method 1500 includes outputting a biometric measurement based on the first sensor data and the second sensor data at the acceptable position.

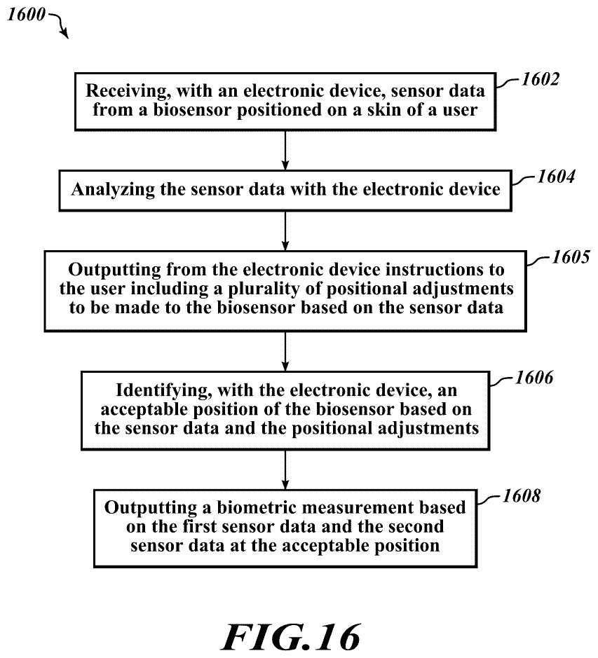

FIG. 16 is a flow diagram of a method 1600, according to one embodiment. The process 1600 can utilize systems, components, and processes described in relation to FIGS. 1-14. At 1602, the method 1600 includes receiving, with an electronic device, sensor data from a biosensor positioned on a skin of a user. At 1604, the method 1600 includes analyzing the sensor data with the electronic device. At 1606, the method 1600 includes outputting from the electronic device instructions to the user including a plurality of positional adjustments to be made to the biosensor based on the sensor data. At 1608, the method 1600 includes identifying, with the electronic device, an acceptable position of the biosensor based on the sensor data and the positional adjustments. At 1610, the method 1600 includes outputting a biometric measurement based on the first sensor data and the second sensor data at the acceptable position.

In one embodiment, a method includes generating first sensor data and second sensor data with a first inertial measurement unit and a second inertial measurement unit of a biosensor device positioned on a skin of a user, transmitting the first sensor data and the second sensor data to an electronic device, and analyzing the first sensor data and second sensor data with the electronic device. The method includes identifying, with the electronic device, an acceptable position of the biosensor device by instructing the user to make a plurality of positional adjustments of the patch based on the first sensor data and the second sensor data and outputting a biometric measurement based on the first sensor data and the second sensor data at the acceptable position.

In one embodiment, a method includes receiving, with an electronic device, sensor data from a biosensor positioned on a skin of a user, analyzing the sensor data with the electronic device, and outputting from the electronic device instructions to the user including a plurality of positional adjustments to be made to the biosensor based on the sensor data. The method includes identifying, with the electronic device, an acceptable position of the biosensor based on the sensor data and the positional adjustments and outputting a biometric measurement based on the first sensor data and the second sensor data at the acceptable position.

In one embodiment, a blood pressure monitoring device includes a sensor housing, a first inertial measurement unit in the sensor housing, and a second inertial measurement unit in the sensor housing spaced apart from the first inertial measurement unit by a fixed distance. The device includes an adjustment mechanism mechanically coupled to the sensor housing and configured to rotate the sensor housing around a first axis and to translate the sensor housing along a second axis transverse to the first axis.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
generating first sensor data and second sensor data with a first inertial measurement unit and a second inertial measurement unit of a biosensor device positioned on a skin of a user;
transmitting the first sensor data and the second sensor data to an electronic device;
analyzing the first sensor data and second sensor data with the electronic device;
identifying, with the electronic device, an acceptable position of the biosensor device by instructing the user to make a plurality of positional adjustments of the patch based on the first sensor data and the second sensor data; and
outputting a biometric measurement based on the first sensor data and the second sensor data at the acceptable position.

2. The method of claim 1, wherein the biosensor device is a blood pressure monitoring device.

3. The method of claim 2, wherein analyzing the first sensor data and the second sensor data includes generating a mean power spectral density correlation value based on the first sensor data and the second sensor data.

4. The method of claim 3, wherein analyzing the first sensor data generating a first mean spectral amplitude average value based on the first sensor data and generating a second mean spectral amplitude average value based on the first sensor data.

5. The method of claim 4, further comprising determining whether a current position of the blood sensor monitoring device is better aligned than a previous position by comparing a sum of the first and second mean spectral amplitude average values at the current position to the sum of the first and second mean spectral amplitude average values at the previous position.

6. The method of claim 5, further comprising determining whether a current position of the blood sensor monitoring device is better aligned than a previous position by comparing the mean power spectral density correlation value at the current position to the mean power spectral density correlation value at the previous position.

7. The method of claim 2, wherein the biometric measurement is a blood pressure measurement.

8. The method of claim 7, further comprising generating the blood pressure measurement by processing the first and second sensor data with an analysis model trained with a machine learning process.

9. The method of claim 1, wherein the electronic device is a smart phone.

10. The method of claim 9, wherein instructing the user includes displaying images or text on a display of the smartphone.

11. The method of claim 9, wherein instructing the user includes outputting audible instructions with a speaker of the smartphone.

12. A method, comprising:
receiving, with an electronic device, sensor data from a biosensor positioned on a skin of a user;
analyzing the sensor data with the electronic device;
outputting from the electronic device instructions to the user including a plurality of positional adjustments to be made to the biosensor based on the sensor data;
identifying, with the electronic device, an acceptable position of the biosensor based on the sensor data and the positional adjustments; and
outputting a biometric measurement based on the first sensor data and the second sensor data at the acceptable position.

13. The method of claim 12, wherein the biosensor is blood pressure measurement positioned adjacent to a carotid artery of the user.

14. The method of claim 12, further comprising:
receiving sensor data after each of the plurality of positional adjustments; and
analyzing the sensor data after each of the plurality of positional adjustments.

15. The method of claim 14, wherein outputting instructions to the user includes displaying images indicating manual adjustments to be made to an adjustment mechanism of the blood pressure monitoring device.

16. The method of claim 12, further comprising:
generating a first mean spectral amplitude average value based on the sensor data; and
generating a mean power spectral density correlation value based on the sensor data.

17. A blood pressure monitoring device, comprising:
a sensor housing:
a first inertial measurement unit in the sensor housing; and
a second inertial measurement unit in the sensor housing spaced apart from the first inertial measurement unit by a fixed distance; and
an adjustment mechanism mechanically coupled to the sensor housing and configured to rotate the sensor housing around a first axis and to translate the sensor housing along a second axis transverse to the first axis.

18. The blood pressure monitoring device of claim 17, comprising:
a main housing; and
control circuitry in the main housing communicatively coupled to the first and second inertial measurement units, wherein the adjustment mechanism rotates and translates the sensor housing relative to the main housing.

19. The blood pressure monitoring device of claim 18, further comprising a wireless transmitter in the main housing and configured to wireless transmit sensor data from the first and second inertial measurement units to an electronic device.

20. The blood pressure monitoring device of claim 18, comprising a strap coupled to the main housing and including a first adhesive region on a first end of the strap and a second adhesive region on a second end of the strap, the main housing being positioned between the first adhesive region and the second adhesive region.
